(19) United States
(12) Patent Application Publication
(10) Pub. No.: US 2022/0079165 A1
(43) Pub. Date: Mar. 17, 2022

(54) FORMULATION FOR PROTECTION AGAINST KIWI BACTERIOSIS, CAUSED BY THE BACTERIUM PSEUDOMONAS SYRINGAE PV. ACTINIDIAE (PSA)

(71) Applicant: UNIVERSIDAD DE CONCEPCION, Concepcion (CL)

(72) Inventors: Ernesto MOYA ELIZONDO, Concepcion (CL); Braulio RUIZ SEPULVEDA, Concepcion (CL); Juan SAN MARTIN MEDINA, Concepcion (CL)

(21) Appl. No.: 17/420,122

(22) PCT Filed: Dec. 19, 2019
(86) PCT No.: PCT/CL2019/050145

§ 371 Date: Jun. 30, 2021

(30) Foreign Application Priority Data
Dec. 31, 2018 (CL) 3895-2018

(51) Int. Cl. A01N 63/27; A01P 1/00; C12N 1/20

(52) U.S. Cl. CPC A01N 63/27; C12R 2001/38; C12N 1/205; A01P 1/00

(57) ABSTRACT

A formulation to protect against kiwi bacteriosis, caused by the bacterium Pseudomonas syringae pv. actinidiae (Psa), having the 5 biomass of the strains Pseudomona protegens ChC7 (RGM 2328) and CA2 (RGM 2329), plus excipients. This formulation enables direct protection against the pathogenic bacterium by activating defense genes present in plants and colonizing their interior, reducing the systemic damage caused by Psa and enabling the generation of protection during crop growth, especially during flowering.

### TECHNICAL SECTOR

The present invention is related to agroindustry, in particular with the Kiwi cultivation industry, this invention generates protection against Pseudomonas syringae pv. actinidiae (Psa) during plant development, especially during flowering.

### BACKGROUND

Today the problem of kiwi bacteriosis (Actinidia chinensis or A. deliciosa) caused by Pseudomonas syringae pv. actinidiae (Psa) remains being current nationally and internationally. This disease generates severe losses in this industry, since it causes necrotic spots on leaves, death of branches, floral abortion and a systemic infection that leads to the death of the plant.

In Chile, according to the background provided by SAG and the Kiwi Committee, it went from 57 orchards that were officially diagnosed with the disease in 2012 to 215 orchards as of Aug. 17, 2016, with 90% of the orchards concentrated in the Maule and Biobio Region, which has meant going from around 720 ha infected with the disease to 2,272 ha, which is currently equivalent to around 20% of the area dedicated to kiwi cultivation in Chile. In the same way, from 2012 to date the disease was focused on the Maule and Biobio Region, but in 2014 it reached the O'Higgins region and in 2015 it occurred affecting an orchard in the Melipilla commune, Metropolitan Region.

Given the climatic conditions that have prevailed during these last springs, yellow kiwi (Actinidia chinensis) orchards, which had 7% of plants with infection symptoms of the disease in autumn, about 40% of plants with disease symptoms in spring. In 2015, kiwi orchards, both green and yellow, showed a high incidence of flower buds with necrosis and leaf damage, which generated losses of up to 60% of production in organic kiwi producers during the 2015-2016 production season. During the 2018-2019 season, the conditions for infection for Psa were excessively favorable, leading producers to production losses between 60 and 70%, due to the recurrent spring rains that occurred between the months of September and November, which favored symptoms such as “red weeping”, necrosis of flower buds and leaf spots, which is leading many producers to uproot this species. This situation has the kiwi industry in a situation of status quo, since there are no new plantations of this fruit tree and there is little interest in planting it due to the problems that its management implies, which increases its costs, and the risks of losses that its production brings.

The problem continues, since the strategies for the control of kiwi bacteriosis caused by Psa are based on the application of products based on copper salts, however, these products have the limitation that they cannot be used in the flowering period, due to the phytotoxicity that they cause in the flower and the damage that they can cause to bees, for which the kiwi plant is unprotected in the period in which the performance of the orchard is defined. In addition, the different products that contain copper do not control the disease, but only keep it at a limit that ensures less loss, and additionally the repetitive and indiscriminate use of copper products can quickly lead to the appearance of bacterial strains resistant to copper, as has been recently shown (Colombi et al. 2017). Recent studies conducted in New Zealand show that there is an evolution of resistance to copper in Psa isolates through the acquisition of conjugative and plasmids elements, which represent 25% of the isolates obtained in a sampling carried out between 2015 and 2016 (Colombi et al., 2017).

On the other hand, the use of antibiotics and chemical resistance inducers such as acibenzolar-S-Methyl (Bion® or Actigard®) allow covering critical phenological periods of the plant, but they are not recommended to be used in organic farms, which in Chile they are the ones that show the highest profitability. Antibiotics for use in agriculture are prohibited in different destination markets for export kiwi, and the use of antibiotics and chemical resistance inducers such as acibenzolar-S-methyl cannot be used in flowering.

Currently, two biological products have entered the Chilean market, one that acts by competition of infection sites (Nacillus®, from Bio Insumos Nativa SPA.) and another that has antagonistic activity based on Bacillus subtilis QST-713 strain (Serenade, Bayer SA), in addition to the chemical resistance inducer based on acibenzolar-S-methyl (BION® 50WG, Syngenta SA).

In this context, the development of biological resistance inducers that can cover the critical moment that is generated in organic kiwi farms, where antibiotics or a chemical resistance inducer cannot be used, is a necessity in the kiwi worldwide production industry.

Given this need for resistance elicitors for kiwi worldwide farms, different bacterial strains producing 2,4-DAPG, pioluteorin and pyrrolnitrin, which are capable of acting by direct antibiosis on Psa, have been evaluated; induce the expression of a series of genes associated with the defense mechanisms of the kiwi plant and, in a preliminary way, observe that it can endophytically colonize the kiwi plant, which has been associated with a lower expression of symptoms of the disease, counteracting the infection caused by Psa in periods or farms where chemical treatments cannot be used or cause a drop in yields.

Patents related to the use of bacteria that produce 2,4-DAPG are associated with the treatment of seeds for the control of fungi that affect the roots of wheat or grass or the biosynthesis of the compounds produced by these bacteria or mechanisms to evaluate the activation of genes associated with the production or expression of genes in this type of bacteria.

Finally, it is worth mentioning that the large transnational agrochemical companies have been investing large sums in developing biological alternatives for the management of plant diseases and pests, which is why the development of biopesticide has become one of the most dynamic industries in the last years.

Some patents covering the use of resistance elicitor strains are:
1. Patent application WO2013/121248 Novel Pseudomonas fluorescens strain and uses thereof in the biological control of bacterial or fungal diseases, in which a strain of P. fluorescens, deposited under the registration number DSM 25556, which acts as a biological control against bacterial and fungal pathogenic strains, prevention method and a formulation are also disclosed.
2. Patent application WO2010/037072 Pseudomonas bacterium, from Novozymes AS (Denmark). A new strain of Pseudomonas deposited under registration number DSM 21663 is introduced. Which promotes plant growth and produces antibiotics such as 2,4-diacetylphloroglucinol (DAPG); pyrrolnitrine (PRN) and indole-3-acetic acid (IAA). The strain has the ability to suppress the action of pathogens of bacterial and fungal origin.
3. Invention patent KR20100115659 Antibiotics for bacterial canker of citrus from drug resistance Pseudomonas aeruginosa No3 and antibiotics, and its application, from the Industry Foundation of Chonnam National University, Korea. This document discloses a strain of Pseudomonas deposited with the registration number KFCC11444P which produces antibiotic substances, such as linoleic acid, against the pathogens that produce canker such as Xanthomonas citri or Pseudomonas syringae pv actinidiae. In addition, a second strain is filed, which was obtained through hybridization with transposon miniTn5, this strain deposited with the number KFCC11445P, shows improved features for the same purpose.
4. Patent application WO2018/047123 Biological control of plant pathogenic microorganisms, of The New Zealand institute for plant and food research limited. This application discloses the use of a yeast Aureobasidium pullulans, called YBCA5, as a biocontroller of bacteria of the species Pseudomonas spp. among which is mentioned Pseudomonas syringae pv actinidiae, also indicating its use in Kiwi plants.
5. Patent application CL201603401 Wild bacterial strain Pseudomonas protens CA02 as a bacterial biofungicide for the control of Botrytis cinerea and other phytopathogenic fungi, from the University of Santiago de Chile. A strain with registration number RGM 2342 is disclosed which is used for the control of fungal infections caused by phytopathogenic fungi in plants.
6. Bio-Protection Research Centre de New Zealand information brochure Desktop evaluation on commercially available microbial-based products for control or suppression of Pseudomonas syringae pv. actinidiae (Stewart et al. 2011) et al.) This article is a review of current products to regulate kiwi ulcer canker by biocontrol, using strains of different genera and species. Among these is the product BlightBan® A506 (NuFarm Inc, USA) which contains the strain Pseudomonas fluorescens A506, which has been studied to control different infectious outbreaks in plants, including the one generated by the strain Pseudomonas syringae pv. actinidiae.

Regarding changes in the regulations, given the high dispersion of Psa in the Maule and Biobio Regions, it has led the SAG to gradually reduce the demands to contain the disease that existed at the beginning, but there is still the desire to maintain the disease outside the Valparaiso and Metropolitan Regions. Among these changes that will enter into public consultation, are the authorization to maintain diseased plants within the properties, before a reduction of the sections of diseased tissue present in the plant, and the possibility of using pollen from the same property in a positive property. The above, that favors the productive costs for the producer, can increase the inoculum conditions within the orchard, for which the use of management and control strategies in kiwi are still necessary, so the policy recommended by the SAG and the Committee of the kiwi is trying to coexist with the disease, which implies continuing to investigate the matter and developing new control alternatives. In the same way, the option of ending the official control is being studied, in the regions where the disease is more widespread, for which the management will be the responsibility of the producers, who must ensure that their productions coexist, being economically viable with the presence of the illness.

### BRIEF DESCRIPTION OF THE DRAWINGS

FIG. 1: Scheme of the different forms of action of the formulation in kiwi plants.

FIG. 2. Inhibition halos observed on a bacterial mat of Pseudomonas syringae pv. actinidiae strain 105743 generated by Chilean isolates of Pseudomonas protens who possess the gene phlD+ associated with the production of 2,4-DAPG and the antibiotic streptomycin (Ant.). KB: King B broth medium solution.

FIG. 3. Inhibition radius showed by Chilean bacterial colonies that possess genes associated with the production of 2,4 DAPG on colonies of Pseudomonas syringae pv. actinidiae after 48 hours of co-cultivation.

FIG. 4. Area under the disease progress curve (AUDPC) in leaves of seedlings of Actinidia deliciosa (green kiwi) after 30 days of having been inoculated with PSA and treated with different Chilean isolates of Pseudomonas protens who possess the phlD+ gene associated with the production of 2,4-DAPG and the antibiotic product Streptoplus®. Control: Not inoculated plant with PSA. Both experiments were carried out under equal conditions, with a difference of three months between them. Different letters on each bar indicate significant differences (α=0.05) according to a non-parametric test by Kruskal Wallis.

FIG. 5. Damage severity observed on the third leaf in Actinidia deliciosa seedlings inoculated with Pseudomonas syringae pv. actinidiae (a), and co-inoculated with Pseudomonas protens Ca2 strain (b) and ChC7 strain, after 8, 20 and 30 days (left to right) of the inoculation with the phytopathogenic bacteria.

FIG. 6. Plants of Actinidia deliciosa after 21 days of being inoculated with Psa. Only one treated with water (left) and another induced with strain Pseudomonas protens Ca2 strain (right).

FIG. 7. Resistance induction effect of treatments applied to foliage based on acibenzolar-S-methyl (Bion® 50WG) and two bacteria Pseudomonas protens strains in the control of Psa in green kiwi (Actinidia deliciosa) expressed as integration in the area under the disease progress curve (AUDPC) after 30 days of evaluation.

FIG. 8. Resistance induction effect of treatments applied to foliage based on acibenzolar-S-methyl (Bion® 50WG) and two bacteria Pseudomonas protens strains in the control of Psa in green kiwi (Actinidia deliciosa) expressed as the number of necrotic spots on the leaves on three evaluation dates after being inoculated with Psa except for the control.

FIG. 9. Resistance induction effect of treatments applied to root based on acibenzolar-S-methyl (Bion® 50WG) and two bacteria Pseudomonas protens strains in the control of Psa in green kiwi (Actinidia deliciosa) expressed as integration in the area under the disease progress curve (AUDPC) after 30 days of evaluation.

FIG. 10. Resistance induction effect of treatments applied to root based on acibenzolar-S-methyl (Bion® 50WG) and two bacteria Pseudomonas protens strains in the control of Psa in green kiwi (Actinidia deliciosa) expressed as the number of necrotic spots on the leaves on three evaluation dates after being inoculated with Psa except for the control.

FIG. 11. Relative expression of 10 defense genes in seedlings of Actinidia deliciosa in plants inoculated to the foliage (H) or to the root (R) with the antagonist bacteria and the chemical inducer Acibenzolar-S-methyl (Bion 50 WP, in a dose of 0.2 g L⁻¹) after 1 day (1 D), 7 days (7 D) and 15 days (15 D) after spraying, for each gene compared to its uninoculated control.

FIGS. 12A-12B. Relative expression of 4 defense genes in seedlings of A. chinensis in plants inoculated to the foliage with bacteria Pseudomonas protens, Pantoea spp. (AB411 and AP113 strains), the chemical inducer Bion® 50 WP, in doses of 0.2 g L⁻¹, and P. syringae pv. actinidiae (PSA) after 1 (FIG. 12A) and 7 (FIG. 12B) days after spraying, compared to its uninoculated control.

FIG. 13. Percentage of damage severity observed after 7 days on leaves of 2-year-old plants of kiwi cv. Hayward that had been treated to foliage twice with Pseudomonas protens and other treatments in San Carlos, Ñuble Region, during the 2016-2017 season.

FIG. 14. Number of necrotic spots observed after 7 days on leaves of adult plants of kiwi cv. Hayward that had been treated to foliage twice with Pseudomonas protens and other treatments in San Carlos, Ñuble Region, during the 2016-2017 season.

### DETAILED DESCRIPTION

The present technology corresponds to a formulation based on Pseudomona protegens strains which allows protection against kiwi bacteriosis, caused by the bacteria Pseudomonas syringae pv. actinidiae (Psa). This formulation, when sprayed in different periods of plant development, can directly protect against bacteria, activate defense genes present in plants, which translates into a systemic signal that will protect the entire plant for around 7 to 14 days, and will colonize the interior of the same reducing the systemic damage that Psa generates, allowing to generate protection during the development of the crop, especially during flowering, which will reduce the damage caused by the disease, and that will finally translate into higher yields in the orchard.

Strains used in the formulation correspond to Pseudomona protegens ChC7 strain and Pseudomona protegens Ca2 strain, both deposited in the Chilean Collection of Microbial Genetic Resources, under deposit numbers RGM 2328 and RGM 2329 respectively. Both deposits were made on Sep. 30, 2016.

These strains were selected from a group of 48 strains for their features of biocontroller potential on the pathogenic species. This exceptional capacity found in the two chosen strains gives a distinctive feature to the formulation disclosed.

It should be noted that the bacteria that are being worked on in this proposal are genetically sequenced in the 16S ribosomal RNA gene, and correspond to species of Pseudomonas protens that have mutations in their genomic sequence that make them different from other species described in the gene sequence banks existing worldwide. On the other hand, these strains have demonstrated the ability to solubilize immobilized phosphorus, produce indole acetic acid and other biosurfactant properties, which are not common features of isolates of the same species, such as P. protens Pf5 strain isolated in the United States (Paulsen et al., 2005; Ramette et al., 2011). Similarly, Chilean strains inhibit Psa under conditions in vitro, while the Pf5 strain does not.

The formulation comprises a liquid bacterial suspension >107 CFU mL-1 obtained from the fermentation of bacterial strains in King B (KB) liquid medium for 24 hours at 24° C. (75.2° F.) and 150 rpm of stirring. For its preparation, an aliquot of 10 μL of the original strain of the bacteria is taken, which is kept in glycerol 20% at −80° C. (−112° F.), and is placed to grow in 10 mL of KB medium for 24 hours and then inoculated with 2 mL of this suspension (˜107 CFU mL⁻¹) per 100 mL.

This formulation has proven effectiveness in controlling Psa when applied in concentrations between 6% and 50% diluted in water. It must be prepared fresh for each application, but can be kept refrigerated at 4° C. (39.2° F.) for 5 to 7 days, in which the bacteria survive.

The tests carried out with the formulation in farms with green kiwi show bacteriosis control levels close to 50% when applied prior to the flowering period, these results are similar to those obtained by the resistance inducer acibenzolar-S-methyl (ASM), or the use of copper hydroxide.

This formulation or bioproduct can be applied alone or in combination with other biocontrollers, whose form of action is competition for spaces, improving control efficiency and differentiating itself from other products on the market. For example, its application in autumn, prior to leaf fall, allows covering the wounds left by the leaves when falling and that are a source of income for Psa and other phytopathogenic bacteria of Pseudomonas genus, favoring endophytic colonization in twigs and trunk of adult plants during winter. At the beginning of sprouting, it can be reapplied to favor endophytic colonization of the new tissue that is being formed and activate resistance genes in the plant that inhibit Psa infection, to continue with applications of copper or in alternation with products in copper base until prior to flowering, to end with applications during flowering, to reduce the damage that occurs at that time, just when cupric bactericides, ASM and antibiotics cannot be used, and when considerable losses have been observed in years with prevailing spring conditions for the development of the disease. Unlike other products, our formulation is presented as a resistance inducer and producer of antimicrobial compounds that inhibit the development of Psa.

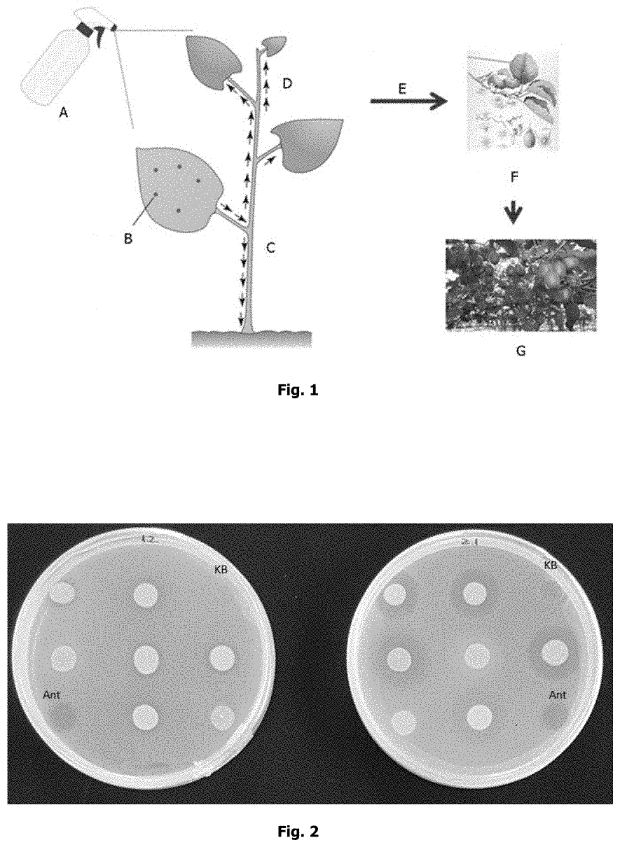

FIG. 1 shows the action model of the formulation when used together with another biocontroller, in A) the formulation is presented in combination with another product; B) spraying this formulation allows: direct antibiosis, resistance induction, endophytic colonization and competition for tissues; C) the generated signal is transported through the plant; D) this generates a Systemic Acquired Resistance (SAR); E) which results in inhibition of Psa infection; F) there is protection during the flowering period; G) which translates into higher yields and less incidence of PSA in organic and conventional orchards.

The application recommendation of this product, due to its protection features, should be according to the following model:
a) In autumn: post-harvest application, allows endophytic colonization, and application during leaf fall, allows protection of wounds occurred during this process.
b) In winter: application of the product that allows the colonization of the strains on the trunk.
c) In spring: i) application at the beginning of the sprout, this allows the colonization of the new tissue; ii) alternate applications during vegetative growth, allows antibiosis and induction of resistance; iii) application in flowering, allows the protection of the flower.

### APPLICATION EXAMPLES

### Example 1: Selection of Beneficial Strains for the Elaboration of a Bioproduct Against Pseudomonas syringae pv. Actinidiae (Psa)

1.1 Evaluation In Vitro of Direct Activity of the Beneficial Strains on Pseudomonas syringae pv. Actinidiae (Psa).

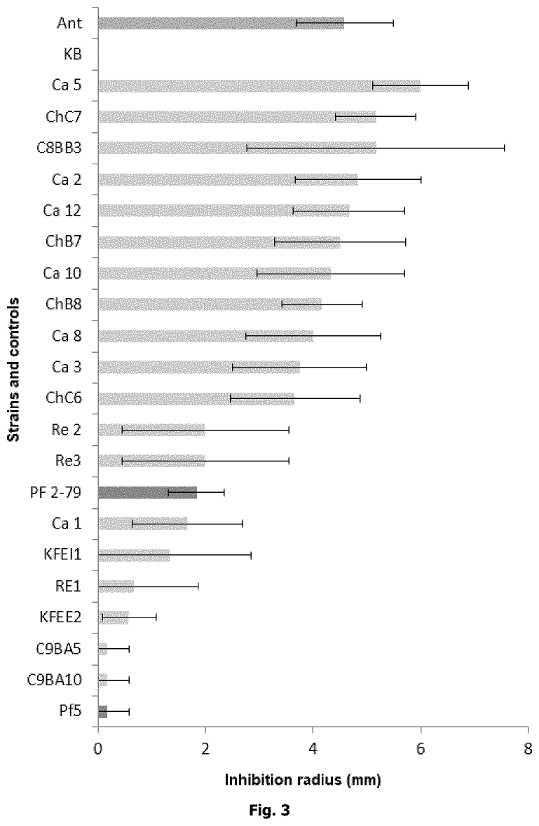

Seedings of PSA bacterial tapestries or lawns were used, which were carried out using 100 and 500 μL of a bacterial suspension applied and spread on the Petri dish with King B agar medium. On the grass, equidistant colonies of 2 to 5 μL of the bacteria under study were placed and the radius of inhibition halo generated by the bacteria or the inhibition area generated using the Image J image processing software was evaluated, after 48 h of incubation at 24° C. (75.2° F.). These studies were carried out with 48 bacterial strains, they were repeated twice and a completely randomized design was used. Comparison of groups of strains were analyzed using Ward's clustering method, and variance analysis within each group and comparison of means using the Tukey or LSD test. Of the 48 evaluated strains, 21 showed different degrees of inhibition over Psa bacterial growth in King B medium (example of plates used, FIG. 2). Of these, 18 are Chilean bacteria that present the phlD gene, associated with the production of 2,4-DAPG, and that developed halos or inhibition areas similar to those generated by an antibiotic such as streptomycin, as can be seen in the inhibition radius graph (FIG. 3). Bacteria with the highest inhibition in vitro on Psa were P. protens Ca2, ChC7, Ca5 and C8BB3, which were selected for the antibiosis control tests in kiwi seedlings. Bacteria of the genus Pantoea that had been proposed for having chitinolytic and glucanolytic activity did not show antimicrobial activity on the bacteria.

### 1.2 Evaluation of Control of Psa in Kiwi Seedlings to Determine the Control Effect of Pseudomonas Producing 2,4-DAPG.

Authorization was obtained from the SAG to work with the Psa bacteria and a protocol was established using kiwi seeds. A. delicious var. Hayward that allowed to obtain kiwi plants with 5 non-cotyledonous leaves in 3 to 4 months to perform evaluation tests with the Psa strain 105743 obtained by the SAG.

Seeds were treated with a 2500 ppm solution of gibberellins (Giberplus® SP, Anasac) for 24 h prior to sowing in Petri dishes with water agar, where they were kept at room temperature until germination. Once germinated, they were transplanted to trays with peat and perlite substrate in a 2:1 w/w ratio, to which 4 gL⁻¹ of the fertilizer mono granule of prolonged release Multicote (Anasac), remaining in the trays until reaching its third leaf, to be transplanted into 1000 cm³ (61.02 in³) pots. The plants during their development were kept in a growth room under controlled conditions of constant temperature of 20° C. (68° F.) and photoperiod of 14 hours of light: 10 hours of darkness delivered by artificial LED light in red, blue, yellow and white wavelengths.

Two groups of experiments were developed to determine the antibiosis effect of beneficial bacteria on Psa and another to determine the induction resistance effect when applying beneficial bacteria to control the disease. In the antibiosis test the plants were co-inoculated with Psa and the P. protens Ca5, Ca2, ChC7 strains and P. fluorescens C8BB3 strain were evaluated.

The evaluation of the ability to induce resistance of the Ca2 and ChC7 strains when inoculated into the rhizosphere was carried out through a two mL Eppendorf tube with seven holes that was buried in the substrate in the center of the pot, where each system radicular was inoculated with 10 mL of the bacterial suspension at a concentration greater than 10⁸ CFU mL⁻¹, ASM in the described dose or with sterile distilled water.

In both inoculation systems, six days after pre-treatment, the pathogenic bacteria were inoculated by spraying eight mL of the bacterial suspension at a concentration greater than 10⁸ CFU mL⁻¹ on all the foliage of the plant. Twenty-four hours before and after the inoculation of the antagonistic bacteria and PSA, to the foliage, the plants were kept in an environment of 80% relative humidity and 20° C. (68° F.), in order to encourage the opening of the stomata in order to facilitate the entry of bacteria into plants.

In antibiosis in plants and induction resistance experiments, the count of necrotic spots per leaf was considered and a visual estimate was made of the percentage of the affected leaf area by the disease. In the first antibiosis experiment, it was evaluated at 8, 20 and 30 days, while in the second test and in those of induction resistance it was evaluated at 7, 14 and 21 days.

Treatments were arranged in a completely randomized design within the growth room, considering four repetitions. With percentage values of damage in leaves, the area under the disease progress curve (AUDPC) was calculated, which represents the accumulated damage value achieved by the plants throughout the three evaluations carried out. Data on the number of spots was transformed to Log₁₀ (data+1). Transformed data of the number of spots and the AUDPC data were analyzed regarding the normality of their distribution using the Shapiro-Wilk normality test and homoscedasticity test, and then an ANOVA was applied, if the assumptions were met, and if there were significant differences (P≤0.05), a comparison of means was made with the LSD Fisher test (α=0.05). If the assumptions of normality and homogeneity of variances were not fulfilled, the non-parametric Kruskal-Wallis test was applied, and if there were significant differences (P≤0.05), a comparison of means was performed by pairwise comparison. All statistical tests were performed with the SAS or Infostat statistical software and the Microsoft Excel statistical analysis tool.

The antimicrobial bacteria and Psa were cultivated in King B or LB broth medium at 25° C. (77° F.) for 24 h under stirring at 200 rpm until obtaining bacterial suspensions of a concentration greater than 10⁸ CFU mL⁻¹ of each one, those that were centrifuged for 5 min at 6000 rpm in a centrifuge to eliminate the supernatant and replace it with a saline solution (0.89 g L⁻¹), then each treatment was mixed with Psa in a 1:1 v/v ratio, to be sprayed 14 mL of mixture on each plant. In addition, control treatments as a 1:1 v/v mixture of a suspension of 10⁸ CFU mL⁻¹ of Psa plus a solution of the commercial antibiotic in doses of 50 g hL-1, based on streptomycin sulfate (25% w/w) plus oxytetracycline hydrochloride (3.2% w/w) (Strepto-plus® WP, Anasac SA, Chile) and a 1:1 v/v mixture of a suspension of 10⁸ CFU mL⁻¹ of Psa plus saline were considered. Also, it was considered a baseline treatment to which only saline solution was sprayed.

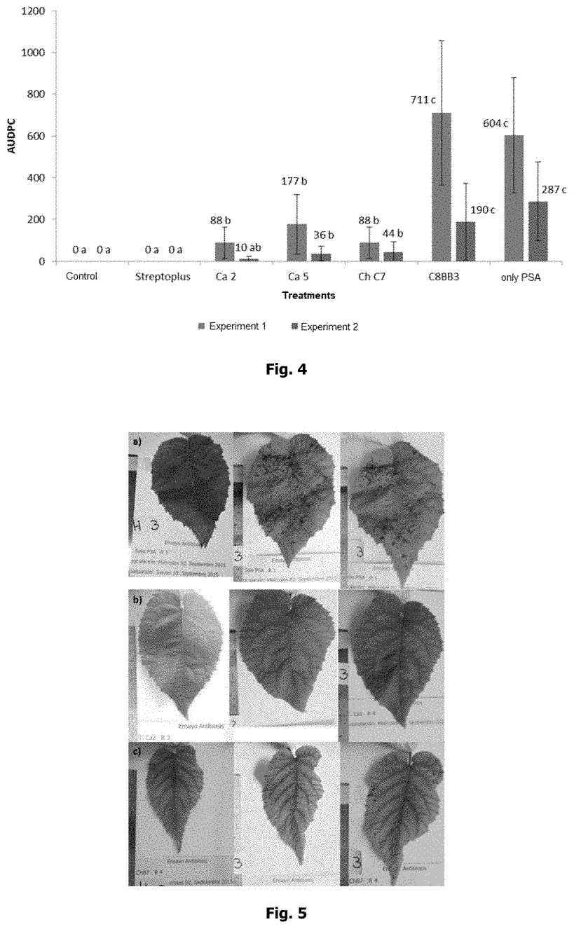

Results shown in FIG. 4 are interesting, Ca2 strain reduced on average between 85.4 and 96.6% the infection level of the accumulated disease in the time between both experiments, and was followed by ChC7 (85.4 and 84.7%) and Ca5 (70.6 and 87.6%). C8BB3 strain did not show control activity on Psa, since it was not different from the inoculated control with this pathogenic bacterium. These values are not statistically different from the use of commercial antibiotic Strepto Plus WP (streptomycin sulfate 25+oxytetracycline hydrochloride 3.2) and from what was observed on not treated plants or inoculated with Psa (P<0.05). Given the good results shown by the Ca2 and ChC7 strains, we continued working with these two strains.

Based on the obtained results, control evaluation based on induction resistance was carried out evaluating the ability of the Ca2, Ca5 and ChC7 strains to protect from Psa infection by inducing resistance when they were applied to the foliage and roots of kiwi plants. Pre-treatment with sterile distilled water and the application of the commercial elicitor acibenzolar-S-methyl (Bion® 50WG, Syngenta, Chile) in the dose of 15 g hL⁻¹ was considered as control treatment. In the foliage treatments, 5 mL were sprayed on the abaxial face of two fully expanded young leaves on each plant, which was carried out in an isolated chamber to avoid drift from bacterial suspensions. Results of these experiments show that after 8 days of inoculating the plants with antagonistic bacteria and Psa, a severity infection level of 12.8% of damage in leaves by Psa was observed, while those treated with Ca2, Ca5 and ChC7, reached infection levels of 0.8, 3.8 and 2.3% on leaves, respectively. (FIG. 5).

1.3 Evaluation in p/Ant of Pseudomonas Strains with the pHlD+ Gene, which is Associated with the Production of 2,4-DAPG, which have Shown Antibiosis Activity on Psa, in their Ability to Induce Resistance for the Psa Control.

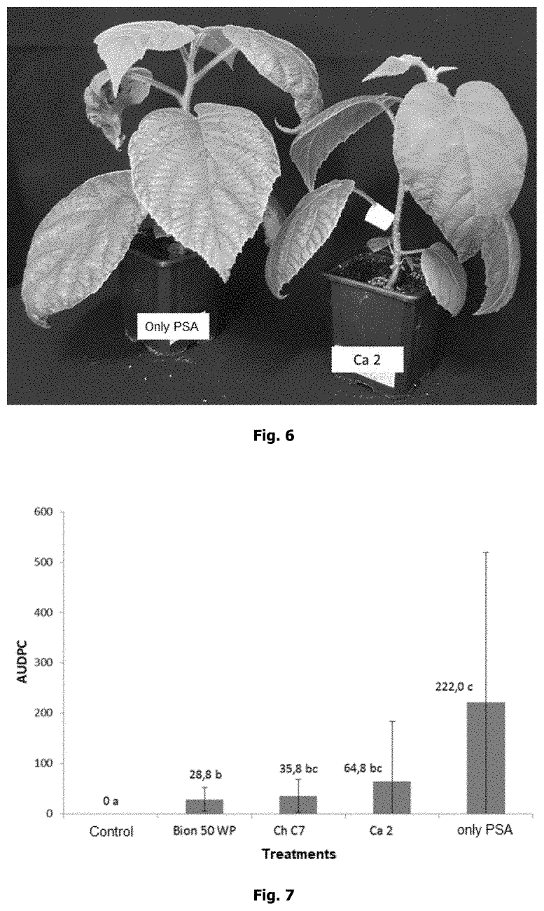

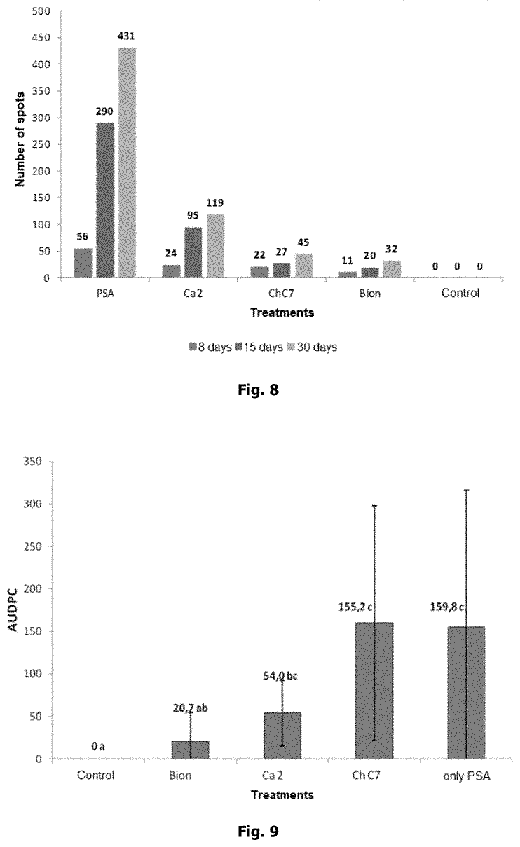

Two experiments were carried out to determine the control effect of the P. protens Ca2 and ChC7 strains, compared to an untreated control and a treated control with acibenzolar-S-methyl (ASM, product BION 50WP, Syngenta), which was used as a standard resistance inducing product for Psa control. This experiment was carried out considering applying the treatments to the foliage and applied to the root, FIG. 6 shows plants 21 days after being inoculated with Psa, on the left one only treated with water and on the right one induced with the Ca2 strain. Considering the area under disease progress curve (AUDPC), in the experiment in which resistance inducers were applied to the foliage, it was observed (FIG. 7) that there were differences between Bion 50 WP (0.2 g L⁻¹) and both antagonist strains with respect to the plants that were only inoculated with Psa 105743 strain, in the first experiment, while in the second the ChC7 strain was not different from the inoculated control or from the other treatments. When considering the number of spots (FIG. 8) there was a greater severity in the first experiment where differences were noted between the treatments with inducers and the inoculated control, since despite not observing statistical differences between ChC7 strain and Bion® 50 WG with respect to the control, these treatments reduced the number of spots on average by 87.5 and 91.0%, respectively. The Ca2 strain was statistically different from the control, but not from the other two treatments and reduced the number of spots by 92.1%. The second experiment had a low number of spots and no differences were observed between treatments, although there was a tendency to reduce these when using resistance inducers in kiwi seedlings.

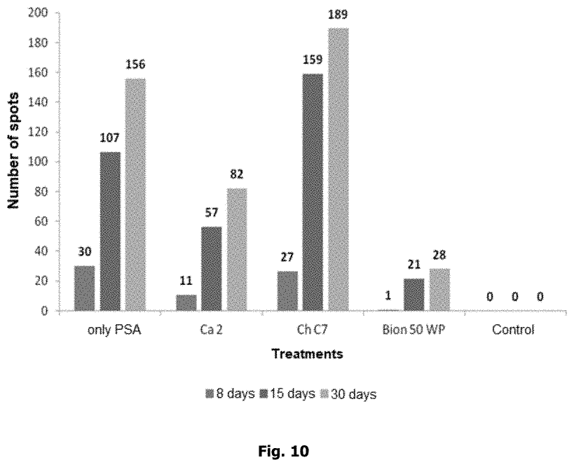

Considering that bacteria from Pseudomonas with phlD gene were isolated from the roots of plants, the induction of resistance in seedlings was evaluated considering inoculating them with a suspension of them in the roots through a tube with 8 holes, which was buried in the substrate in the center of the pot with the seedling, so that after 7 days, inoculate the foliage with Psa. When considering this root treatment methodology, it could be observed (FIG. 9) that the bacteria had a variable behavior in the expression of the disease considering the AUDPC value, since in the first experiment ChC7 was not different from inoculated control with Psa, but in the second experiment it was. For its part, Ca2 strain and Bion® 50 WP in doses of 0.2 g L⁻¹ they were in both experiments different from the treatment only treated with Psa after 30 and 21 days of inoculation with the phytopathogenic bacteria. When considering the number of necrotic spots, no great differences were observed between treatments (FIG. 10), but the same trend was observed for both experiments, reducing on average the ChC7 strain by 88.8% the number of spots on the leaves and the Ca2 strain 47.4 and 73% the expression of the disease in each experiment, respectively, and not being statistically different from the product Bion® 50 WP in both experiments. Again, high variation in the expression of the disease in the treatment only treated with Psa, or the sample size of only 4 plants (repetitions) per treatment, prevented the detection of the tendency to reduce the disease presented by the Ca2 strain. Notwithstanding the foregoing, when considering the average number of necrotic spots on evaluated leaves, it suggests that the use of the Ca2 strain would help to reduce the level of inoculum present on the plants in a period of 30 days.

1.4 Evaluation of Defense Gene Activation in Kiwi Plants Inoculated with Pseudomonas Strains Producing 2,4-DAPG.

Evaluation of defense gene expression in kiwi seedlings by bacteria that were able to reduce Psa infection was carried out considering a comparative evaluation of antagonist strains and Bion® 50WG, in doses of 0.2 g L⁻¹, in their expression of messenger transcriptomes (mRNA) for pr1 genes (Pathogenesis-related protein 1), pr8 (pathogenesis-related protein 8), ICS1 (Isocorismate synthase), PAL (Phenylalanine ammonium lyase) and lox (lipoxygenase) from the work of Cellini et al. (2014), plus kiwi plant-specific starters for genes associated with plant defense: pr4 (Pathogenesis related protein 4), TLP1 (Thaumatin-like protein), APX (L-ascorbate peroxidase), CNP60 (Chaperone-60) and Serpine (Serpin ZX), developed in our laboratory based on the work of Petriccione et al. (2013 and 2014) in kiwi seedlings. These defense genes make up a platform that made it possible to evaluate by qPCR, through the ΔΔCt method, the relative expression of these genes, using the gene Actin as a reference gene. In an experiment that was carried out on green kiwi seedlings and the response of kiwi plants to foliage sprays was evaluated and root applications of the ChC7 and Ca2 strains, Bion 50WP and a water control could be observed, which applied to the foliage, Bion® 50WP had gene expression patterns similar to those generated by the Ca2 strain, increasing the expression of genes pr1, PAL, pr4, TLP1 Y lox. ChC7 strain had a time overexpression of pr4 gene similar to Bion® 50WG. Increased expression of lox gene is associated with responses of systemic acquired resistance (SAR) and peroxidase production, being relatively high in kiwi plants treated with the chemical inducer and both bacteria, the same was observed for pr4 genes, associated with the chitinases and pr1 production, associated with antifungal compounds production and the ICS1 gene, which is associated with the initial stage in the production of salicylic acid, which is the precursor in SAR responses.

Application of the treatments to the root did not result into a higher expression of the 9 genes evaluated compared to the application to the foliage. There was only a significant increase in pr1, TLP1 Y pr8 gene expression, where the Bion® 50WG product had the highest increases in gene expression. P. protens strains had slight increases in TLP1 and ICS1 gene expression, and the Ca2 strain increased the expression of pr8 gene (type III chitinases), one day after application, similar to what was done by the chemical inducer. In TLP1 gene expression, Bion 50WG increased its expression as the days passed since its application to the roots; while ChC7, had a rise on the day after the application to fall to zero expression after 7 days, while Ca2 maintained constant expression during the evaluation period.

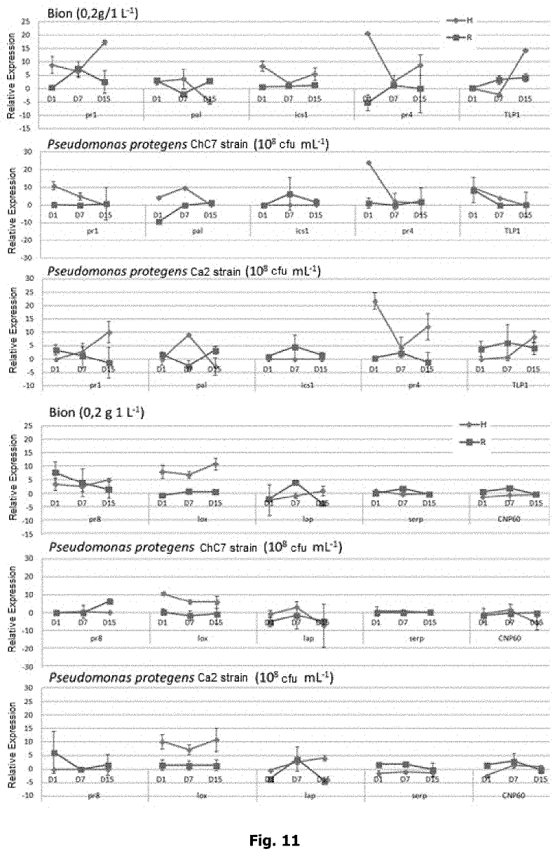

In another experiment, the ability to increase the expression or induce pr1, ICS1, PAL Y TLP1 genes was comparatively evaluated by the P. protens Ca2, Ca6, Ca10, ChB7, and ChC7 strains, Pantoea spp., AB411 and AP113, Bion 50 WP strains in doses of 0.2 g L⁻¹, and PSA 105743 strain and a control not treated with bacteria, after one and seven days after the application of these treatments. In FIG. 11 we can see that the treatments were able to increase the expression of the genes under study after 24 hours of application, highlighting Bion® 50WP that markedly increased the expression of pr1 gene, while Ca2 markedly increased the expression of PAL gene. Psa had low gene expression, increasing TLP1 gene expression, whose expression was not different from that achieved by Ca10 and ChB7 strains. After 7 days of having applied the treatments on the plants, it was observed that Bion® 50 WG increased pr1 gene expression by 60 times, with respect to the control, followed by ChC7, Ca6, PSA and AP113 strain. PAL and ICS1 gene expression were low for all bacteria, while TLP1 gene expression had its highest expression in ChC7, PSA, Ca2, Ca6 and Bion® 50 WG strain.

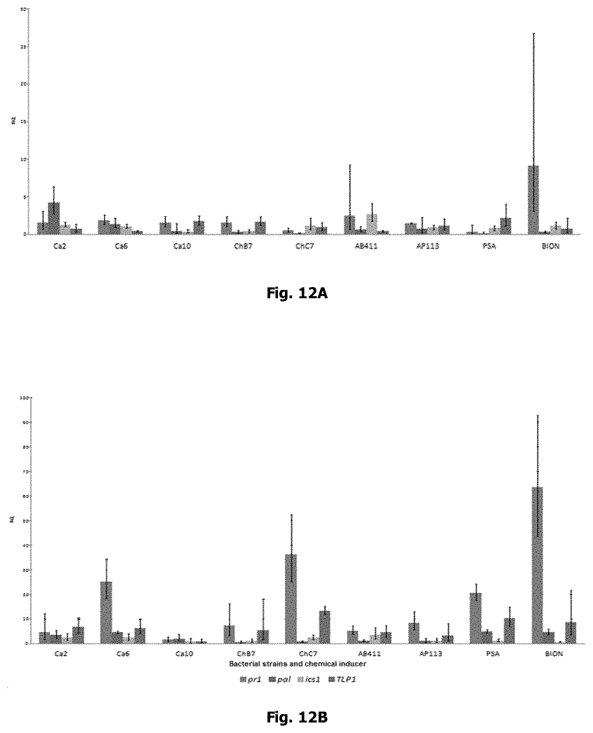

In an experiment with yellow kiwi seedlings (A. chinensis) treated with Psa, Bion® 50WG, Ca2, Ca6, Ca10, and ChC7, and Pantoea spp., AB411 strains after one and seven days after the application of these treatments, an increase in the gene expression of pr1 Y PAL genes was observed (FIGS. 12A-12B) at 24 hours (FIG. 12A), although the expression of these genes was higher in the plants treated with Psa. At 7 days (FIG. 12B) there was an increase in the expression of the four genes evaluated by the bacteria under study.

### Example 2: Preparation of Formulation from Pseudomonas Producing 2,4-DAPG

All the tests described above were carried out with liquid suspensions of bacteria that were grown in King B medium and after 48 hours of growth, they were centrifuged to separate the supernatant.

The survival rate to lyophilizate of Ca10, ChB7, Ca2 and Ca6 strains was evaluated, for this, the bacteria multiplied in King B broth, were concentrated, washed in saline solution, and centrifuged prior to a lyophilization process for 48 h at 65° C. (149° F.) under vacuum until samples reached a powdery appearance. After lyophilization, the sample was suspended in 1 mL of saline solution and CFU mL⁻¹ was determined again for each bacterial strain.

Lyophilization process reduced the Ca10 and ChB7 strains populations by exponential two-thirds, while there were no surviving bacteria in the process for Ca2 and Ca6 strains, therefore a lyophilization process would not be adequate to formulate P. protens strains. To overcome this problem, a 1:1 mixture of 10% w/v peptone and 10% w/v sodium glutamate was evaluated as a cryoprotectant for bacteria. The use of cryoprotectant resulted in an increase in the lyophilization time for the sample to acquire a powdery appearance, and in turn significantly improved the survival of the bacteria when lyophilized after 24 hours, since bacterial populations were only reduced between 100 and 10,000 times. However, survival time was affected, since after 15 days it was not possible to obtain viable bacterial cells. Thus, it can be concluded that the possibility of formulations in lyophilized powders for P. protens strains it would not be a viable alternative as a biopesticide formulation to be developed.

Table 1 shows the survival rate expressed in colony forming units (cfu) per mL of different Pseudomonas protens strains observed before and after a lyophilization process for 48 hours at −65° C. (−85° F.) under vacuum.

TABLE 1
Bacterial survival rate before and after a lyophilization process.

| Bacterial | Pre-lyophilized population | Post-lyophilized population |
|---|---|---|
| strain | (cfu mL⁻¹) | (cfu mL⁻¹) |
| Ca10 | 3.0 × 10¹² | 4.3 × 10⁴ |
| ChB7 | 2.8 × 10¹² | 2.0 × 10⁴ |
| Ca2 | 1.9 × 10¹² | 0.0 |
| Ca6 | 2.0 × 10¹² | 0.0 |

Table 2 shows the survival rate expressed in cfu per mL of different Pseudomonas protens strains treated with a cryoprotectant (1:1 mixture of 10% w/v peptone and 10% w/v sodium glutamate) observed prior to its performance and 24 hours and 15 days after a lyophilization process for 76 hours at −65° C. (−85° F.) under vacuum.

TABLE 2
Bacterial survival rate treated with a cryoprotectant.

| | Pre-lyophilized | Post-lyophilized | |
|---|---|---|---|
| Bacterial | population | population | 15 days after |
| strain | (cfu mL⁻¹) | (cfu mL⁻¹) | lyophilization |
| Ca10 | 3.4 × 10¹⁰ | 2.9 × 10⁸ | 0.0 |
| ChB7 | 1.1 × 10¹⁰ | 9.3 × 10⁶ | 0.0 |
| Ca2 | 2.1 × 10⁹ | 2.0 × 10⁶ | 0.0 |
| Ca6 | 5.4 × 10⁹ | 3.6 × 10⁷ | 0.0 |

Due to these results, it was determined that the best way to apply the strains on the plants is through liquid suspensions.

### Example 3: Evaluation in Commercial Orchards of Strain Control Activity or Bioformulations Based on Pseudomonas Strains Producing 2,4-DAPG

During October 2016, three experiments were established to evaluate two strains in their ability to counteract the natural infection of Psa in two commercial farms that had been diagnosed as positive to Psa. Experiments were established in an organic kiwi cv orchard. Hayward, in the Fundo Roma located in San Carlos, Biobio Region, and the other one in a conventional orchard of green kiwi cv. Summer, located in the Pichingal sector, in the commune of Lontue, Maule Region.

Three experiments were established in each orchard, the first experiment considered evaluating the control effect of the treatments to the natural infection by Psa in kiwi plants of one or two years that were placed at the foot of adult plants. In San Carlos, it was established with healthy kiwi cv plants. 2-year-old Hayward, while in Pichingal, was established with one-year-old plants that were obtained from seeds of kiwi cv. Hayward. Plants were sprinkled with the treatments and the pots containing them were placed under the canopy of an adult female kiwi plant on the row of the orchards. Distance between each pot was one plant on the row, taking care that the pot was under the canopy of a female plant and next to a sprinkler. Each treatment was repeated seven times. The second experiment was carried out using three green and two yellow kiwi plants that had 4 to 5 true leaves, which were in pots, individual and were placed in a plastic container (tray) and hung at the height of the canopy of adult kiwi plants in the same place where the pots of the aforementioned experiment were located. This experiment sought to standardize the micro-climate that exists at this height and increase the possibility of a Psa infection. Each container per treatment had 4 repetitions. The third experiment consisted of treating each adult kiwi plant with the treatments where the pot with the 1 or 2-year-old plant or the seedlings contained in the containers was placed.

The treatments to be applied on the kiwi plants and seedlings consisted of a water control, a treatment with copper hydroxide (Kocide® 2000, Anasac, Chile S.A.) in doses of 250 g hL-1, acibenzolar-S-Methyl (Bion® 50 WG, Syngenta, Chile) in doses of 20 g hL-1, two treatments with P. protens bacteria ChC7 and Ca2 strain applied separately, and a 50% mixture of both bacteria. Bacterial suspensions were obtained by culturing for 48 h at 24° C. (75.2° F.) in King B medium until reaching a population >10⁸ cfu mL¹, and said bacterial fermentation was used in doses 50% diluted in water in the tests in pot on the floor and seedlings in the canopy, while in the applications in adult plants, the bacterial suspension was applied at 2% diluted in water. In San Carlos, as it is an organic orchad, in the experiment with an adult plant, Bion® 50WG was replaced by the Nacillus® WP product in doses of 150 g hL-1.

Treatments were applied three times in the season, on October 7 and 21 and November 4 for the experiment in San Carlos, and on October 14 and 28 and November 11 in the experiment in Pichingal. In the first application, used plants in the experiment with a pot on the floor and with seedlings in the canopy were treated in the greenhouse the day before they were placed in the orchard, while adult plants were treated on the dates above mentioned. Experiment with seedlings in the canopy received only two applications since the plants were placed in the orchard on the second application date on October 21 and 28 for San Carlos and Pichingal, respectively. Also, in the last application carried out, due to being in the state of beginning of flowering, the application of the copper-based products was suspended in the experiment with adult plants. In both experimental sites, 3 adult rows will be occupied, leaving the central row to place the experimental units of each experiment, distributing these in a randomized complete block design throughout the row. The edge rows at both test sites were treated with copper (Kocide® 2000 WP, 250 g hL⁻¹, Anasac Chile), applied in a focused way to each plant in these rows, to avoid a possible drift of the product towards the pots or plants of the three experiments.

Plants of the three experiments in both experimental sites were evaluated prior to the third application and after this last application, leaf damage was evaluated at 7, 14, 21 and 28 days later, by counting necrotic spots on a certain number of leaves or using a damage or severity scale to assess the severity of leaf damage, where 0=no spots (healthy), 1=1 to 3 spots/leaf (incipient), 2=4-10 spots/leaf (mild), 3=11-25 spots/leaf (moderate) and 5=26 or more spots/leaf (severe).

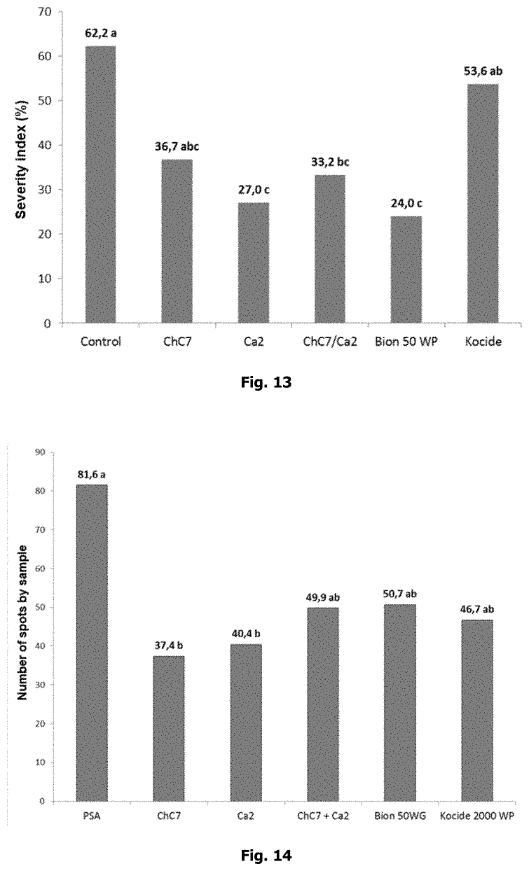

The results allowed observing that in plants in pots at the foot of adult kiwi plants and considering natural infection of the PSA orchard, it was observed in San Carlos that with two applications after 7 days, differences between treatments were observed (P<0.05), presenting the bacterial bioinductors control levels similar to the commercial bioinducer (Bion® 50 WG) and statistically different from the untreated control (FIG. 13). After 14 days, despite observing a lower severity of damage on average, there were no differences between treatments (P=0.112). After three applications, differences were observed between the treatments at 20 and 28 days after the last application, the bioinductors having a better control of the disease after 20 days, being different from the control and not different from copper hydroxide and Bion. 50WG. At 28 days, bioinductors were not different from the control, but neither from the Bion 50WG product. In adult plants, it achieved a reduction of necrotic spots close to 50% with respect to the control (FIG. 14).

1. A strain for the protection against kiwi bacteriosis, caused by Pseudomonas syringae pv. actinidiae (Psa) comprising Pseudomona protegens strain deposited in the Chilean Collection of Microbial Genetic Resources with deposit number RGM 2328.

2. A strain for the protection against kiwi bacteriosis, caused by Pseudomonas syringae pv. actinidiae (Psa) comprising Pseudomona protegens strain deposited in the Chilean Collection of Microbial Genetic Resources with deposit number RGM 2329.

3. (canceled)

4. A formulation for the protection against kiwi bacteriosis, caused by Pseudomonas syringae pv. actinidiae (Psa) bacteria, comprising a liquid bacterial suspension of biomass of Pseudomona protegens RGM 2328 and RGM 2329 strains, in King B medium.

5. The formulation for the protection against kiwi bacteriosis of claim 4, wherein the formulation provides direct protection against the pathogenic bacterium, activating defense genes present in plants and colonizing interior thereof, reducing the systemic damage caused by Psa, enabling the generation of protection during crop growth, especially during flowering.

6. The formulation for the protection against kiwi bacteriosis of claim 4, wherein the formulation is applied alone or in combination with other biocontrollers, whose form of action is competition for spaces.

7. A method of improving control of Pseudomonas syringae pv. actinidiae (Psa) in kiwi comprising applying the formulation according to claim 4 to the kiwi affected by kiwi bacteriosis caused by Psa.

8. The method according to claim 7, wherein the formulation is applied to kiwi seedlings.

9. The method according to claim 7, wherein the formulation is applied to a foliage or root of the kiwi.
